(19) United States
(12) Patent Application Publication
(10) Pub. No.: US 2014/0296713 A1
(43) Pub. Date: Oct. 2, 2014

(54) ULTRASONIC MEASUREMENT APPARATUS, ULTRASONIC IMAGING APPARATUS, AND ULTRASONIC MEASUREMENT METHOD

(71) Applicant: SEIKO EPSON CORPORATION, Tokyo (JP)

(72) Inventor: Takashi HYUGA, Matsumoto (JP)

(73) Assignee: SEIKO EPSON CORPORATION, Tokyo (JP)

(21) Appl. No.: 14/220,372

(22) Filed: Mar. 20, 2014

(30) Foreign Application Priority Data
Mar. 27, 2013 (JP) 2013-065760

(51) Int. Cl. A61B 8/08

(52) U.S. Cl. CPC A61B 8/0858
USPC 600/449

(57) ABSTRACT

An ultrasonic measurement apparatus includes a transceiver unit that controls an ultrasonic transducer device, a processing unit that performs processing for analyzing a body tissue layer of a subject based on a signal received by the transceiver unit, and a notification information output unit that outputs notification information based on a result of the analysis processing. The processing unit performs the analysis processing by specifying a plurality of body tissue layers of the subject, based on a first reception signal of a first timing within a measurement period and a second reception signal of a second timing within the measurement period at which a deformation state of the body tissue layers differs from the deformation state at the first timing, and analyzing thickness information of each of the specified body tissue layers, and the notification information output unit outputs the result of the analysis processing.

### BACKGROUND

1. Technical Field

The present invention relates to an ultrasonic measurement apparatus, an ultrasonic imaging apparatus, an ultrasonic measurement method, and the like.

2. Related Art

Ultrasonic measurement apparatuses that emit ultrasonic waves onto an object and receive reflected waves from interfaces within the object that have different acoustic impedances are attracting attention as apparatuses for use in carrying out internal examination of human subjects. Furthermore, ultrasonic measurement apparatuses are also applied to diagnostic imaging of the surface layers of a subject, such as thickness measurement of the fat and muscle layers and measurement of blood flow.

The invention disclosed in JP-A-4-231945 is one exemplary invention relating to such ultrasonic measurement apparatuses.

Reflection of ultrasonic waves shows up clearly at the boundary between the fat and muscle layers which generally have different tissue structures. However, in the case of a subject who repeatedly diets and rebounds, diet marks remain in the tissue and false boundaries caused by these diet marks show up in ultrasound images (B-mode images). Thus, multiple boundaries appear, hindering interpretation and possibly preventing accurate measurement of the thickness of each body tissue layer.

### SUMMARY

According to some aspects of the invention, an ultrasonic measurement apparatus, an ultrasonic imaging apparatus, an ultrasonic measurement method and the like that are able to improve the analysis accuracy of thickness information of body tissue layers in the case where the subject has false boundaries can be provided.

One aspect of the invention relates to an ultrasonic measurement apparatus including a transceiver unit that controls an ultrasonic transducer device with respect to transmission and reception of ultrasonic waves, a processing unit that performs processing for analyzing a body tissue layer of a subject based on a signal received by the transceiver unit, and a notification information output unit that outputs notification information based on a result of the analysis processing. The processing unit performs the analysis processing by specifying a plurality of body tissue layers of the subject, based on a first reception signal of a first timing within a measurement period and a second reception signal of a second timing within the measurement period at which a deformation state of the body tissue layers differs from the deformation state at the first timing, and analyzing thickness information of each of the specified body tissue layers. Also, the notification information output unit outputs the result of the analysis processing of the thickness information of each body tissue layer as the notification information.

In the one aspect of the invention, the ultrasonic transducer device is controlled with respect to transmission and reception of ultrasonic waves. The body tissue layers of the subject are deformed within a measurement period, and a plurality of the body tissue layers of the subject are specified based on a first reception signal of a first timing within the measurement period and a second reception signal of a second timing within the measurement period. Next, thickness information of each of the specified body tissue layers is analyzed, and a result of having analyzed the thickness information of each body tissue layer is output as notification information.

It is thereby possible to improve the analysis accuracy of the thickness information of the body tissue layers in the case where the subject has false boundaries.

Also, in the one aspect of the invention, the processing unit may derive a group of feature points of the body tissue layers based on the reception signals, and specify the plurality of body tissue layers, based on the displacement information of the feature points between the first timing and the second timing.

It is thereby possible, for instance, to derive feature points as points to be observed in a reception signal, and specify what body tissue layers the different portions of the reception signal represent, based on the displacement information of the feature points.

Also, in the one aspect of the invention, the processing unit may derive first distance information between a first feature point and a second feature point in the group of feature points derived based on the first reception signal, derive second distance information between a third feature point associated with the first feature point and a fourth feature point associated with the second feature point in the group of feature points derived based on the second reception signal, and derive the displacement information of the first feature point, based on difference information between the first distance information and the second distance information.

It is thereby possible, for instance, to derive the amount of change in the positional relationship between an arbitrary feature point and another feature point located peripherally to the arbitrary feature point between the first timing and the second timing as the displacement information of the arbitrary feature point.

Also, in the one aspect of the invention, the processing unit may perform processing for grouping the displacement information of the feature points, and specify the plurality of body tissue layers based on a result of the grouping.

It is thereby possible, for instance, to specify an area in which feature points sorted into the same group are gathered as the extent of an area occupied by one body tissue layer.

Also, in the one aspect of the invention, the processing unit may create a histogram of the displacement information of the feature points, and perform the processing for grouping the displacement information based on the created histogram.

It is thereby possible, for instance, to accurately specify each body tissue layer, even in the case where a luminance peak does not occur at the boundary between body tissue layers.

Also, in the one aspect of the invention, the processing unit may perform processing for associating the feature points in the group of feature points derived based on the first reception signal with the feature points in the group of feature points derived based on the second reception signal, and derive the displacement information of the feature points that are associated in the association processing.

It is thereby possible, for instance, to compute the movement amount of each feature point as the displacement information of the feature point between the first timing and the second timing.

In the one aspect of the invention, the processing unit may derive a first group of feature points of the body tissue layers based on the first reception signal and a second group of feature points of the body tissue layers based on the second reception signal, perform processing for associating the feature points in the first group of feature points with the feature points in the second group of feature points, derive the displacement information of the feature points that are associated in the association processing, perform processing for grouping the derived displacement information and specify the plurality of body tissue layers based on a result of the grouping, and analyze the thickness information of each of the specified body tissue layers.

It is thereby possible, for instance, to improve the analysis accuracy of the thickness information of the body tissue layers in the case where the subject has false boundaries.

Also, in the one aspect of the invention, the notification information output unit may generate, as the notification information, an image representing a first body tissue layer and a second body tissue layer among the plurality of body tissue layers specified by the processing unit with different pixel values.

It is thereby possible, for instance, for a display unit to display an image representing the first body tissue layer and the second body tissue layer with different pixel values.

Also, in the one aspect of the invention, the notification information output unit may generate, as the notification information, an image representing a first body tissue layer and a second body tissue layer among the plurality of body tissue layers specified by the processing unit with different colors or brightnesses.

It is thereby possible, for instance, for a display unit to display an image representing the first body tissue layer and the second body tissue layer with different colors or brightnesses.

Also, in the one aspect of the invention, the notification information output unit may generate, as the notification information, an image representing an amount of change in a thickness of each body tissue layer between the first timing and the second timing.

It is thereby possible, for instance, for the display unit to display an image representing the amount of change in the thickness of each body tissue layer between the first timing and the second timing.

Also, in the one aspect of the invention, the deformation states of the body tissue layers at the first timing and the second timing within the measurement period may differ due to a pressing action of an ultrasonic probe.

It is thereby possible, for instance, to detect change when external pressure is applied to body tissue, with a simple operation.

Also, another aspect of the invention relates to an ultrasonic imaging apparatus including the ultrasonic measurement apparatus and a display unit that displays the notification information.

Yet another aspect of the invention relates to an ultrasonic measurement method including controlling an ultrasonic transducer device with respect to transmission and reception of ultrasonic waves, specifying a plurality of body tissue layers of a subject, based on a first reception signal of a first timing within a measurement period and a second reception signal of a second timing within the measurement period at which a deformation state of the body tissue layers differs from the deformation state at the first timing, analyzing thickness information of each of the specified body tissue layers, and outputting a result of having analyzed the thickness information of each body tissue layer as notification information.

### BRIEF DESCRIPTION OF THE DRAWINGS

The invention will be described with reference to the accompanying drawings, wherein like numbers reference like elements.

FIG. 1 shows an exemplary system configuration of an embodiment of the invention.

FIGS. 2A to 2C show an example of a specific device configuration of an ultrasonic measurement apparatus.

FIGS. 3A and 3B are diagrams illustrating the situation at the time of measurement.

FIG. 4A is a diagram illustrating a B-mode image, and FIG. 4B is a diagram illustrating an A-mode image.

FIGS. 5A to 5C are diagrams illustrating body tissue when under a pressing force and when not under a pressing force.

FIG. 6 is a flowchart illustrating the processing flow in the embodiment.

FIGS. 7A and 7B are diagrams illustrating feature point detection processing.

FIGS. 8A and 8B are diagrams illustrating feature point association processing.

FIGS. 9A and 9B are diagrams illustrating processing for calculating feature point displacement information.

FIG. 10 is a diagram illustrating a displacement information histogram.

FIGS. 11A and 11B are diagrams illustrating a feature point that is noise.

FIG. 12 is a diagram illustrating processing for analyzing thickness information on body tissue layers.

FIGS. 13A and 13B are diagrams illustrating display images.

FIGS. 14A to 14C show an exemplary configuration of an ultrasonic transducer element.

FIG. 15 shows an exemplary configuration of an ultrasonic transducer device.

FIGS. 16A and 16B show an exemplary configuration of ultrasonic transducer elements provided in correspondence to each channel.

### DESCRIPTION OF EXEMPLARY EMBODIMENTS

Hereinafter, an embodiment of the invention will be described. First an outline of the embodiment will be described, followed by a description of an exemplary system configuration of the embodiment. Next, the processing flow of the embodiment will be described in detail using a flowchart, and methods employed in the embodiment will be summarized. Exemplary configurations of an ultrasonic transducer element and an ultrasonic transducer device will then be described. Note that the embodiment described below is not intended to unreasonably limit the contents of the invention set forth in the claims. Also, not all constituent elements described in this embodiment are essential to the invention.

### 1. Outline

Ultrasonic measurement apparatuses that emit ultrasonic waves onto an object and receive reflected waves from interfaces within the object that have different acoustic impedances are attracting attention as apparatuses for use in carrying out internal examination of human subjects. Furthermore, in terms of exemplary applications of ultrasonic measurement apparatuses, there are, for instance, pocket ultrasonic viewers that perform diagnostic imaging of the surface layers of a subject, such as thickness measurement of the fat and muscle layers and measurement of blood flow, and expansion into the healthcare field is anticipated.

The invention disclosed in JP-A-4-231945 is one exemplary invention relating to such ultrasonic measurement apparatuses. A subcutaneous fat display/measurement device disclosed in JP-A-4-231945 is compact and easy to use, and is able to accurately measure the fat layer and perform detailed display of measurement results in a readily understandable manner. Thus, a complete picture of the state of the subject's fat layer can be easily grasped by using this device. Also, with the invention disclosed in JP-A-4-231945, the boundary between the subcutaneous fat layer and another layer can also be detected by detecting the peak value of ultrasonic tomographic information (A-mode waveform).

Reflection of ultrasonic waves shows up clearly at the boundary between the fat and muscle layers which generally have different tissue structures. However, in the case of a subject who repeatedly diets and rebounds, diet marks remain in the tissue and false boundaries caused by these diet marks show up in ultrasound images (B-mode images). Thus, multiple boundaries appear, hindering interpretation and possibly preventing accurate measurement of the thickness of each body tissue layer.

In view of this, the ultrasonic measurement apparatus and the like of this embodiment improve the analysis accuracy of the thickness information of body tissue layers in the case where the subject has false boundaries caused by diet marks or the like. Specifically, a pressing operation using an ultrasonic probe is performed on the measurement site of the subject, based on the body tissue characteristic that the subcutaneous fat layer tends to give way more easily than the muscle layer, and the displacement amount of the feature points in the B-mode images before and after the pressing operation is computed. The respective body tissue layers are specified based on the computed displacement amount and the thickness information of each body tissue layer is analyzed.

### 2. Exemplary System Configuration

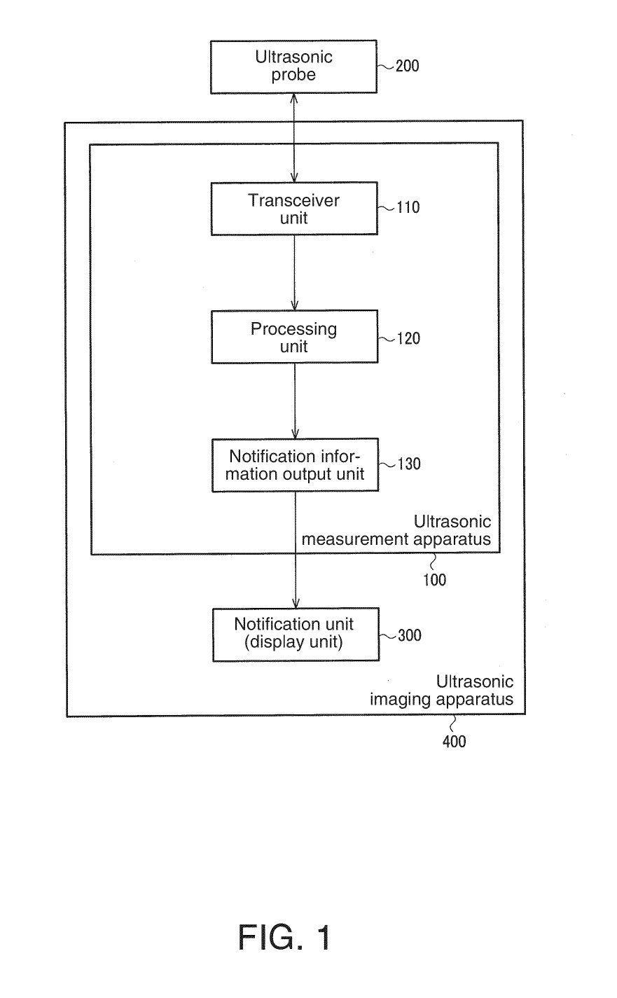

Exemplary configurations of an ultrasonic measurement apparatus 100 and an ultrasonic imaging apparatus 400 of this embodiment are shown in FIG. 1. The ultrasonic measurement apparatus 100 includes a transceiver unit 110, a processing unit 120, and a notification information output unit 130. The ultrasonic imaging apparatus 400 includes the ultrasonic measurement apparatus 100 and a display unit 300. Note that the ultrasonic measurement apparatus 100 and the ultrasonic imaging apparatus 400 including the same are not limited to the configurations shown in FIG. 1, and various modifications can be implemented, such as omitting some of the constituent elements or adding other constituent elements. For example, the ultrasonic measurement apparatus 100 and the ultrasonic imaging apparatus 400 including the same may include a user interface unit (operation unit) for inputting setting values and the like and a storage unit. Also, some or all of the functions of the ultrasonic measurement apparatus 100 and the ultrasonic imaging apparatus 400 including the same of this embodiment may be realized by a server connected by communication means.

Next, processing performed by the respective units will be described.

First, the transceiver unit 110 controls an ultrasonic transducer device with respect to transmission and reception of ultrasonic waves. For example, the transceiver unit 110 includes a transmission pulse generator, a transmission delay circuit, a transmission-reception changeover switch, a reception delay circuit, a filter circuit, a memory, an A/D conversion circuit, and the like.

Specifically, the transmission pulse generator (pulsar circuit) applies a transmission pulse voltage to drive an ultrasonic probe 200.

The transmission delay circuit focuses a transmit beam. For this purpose, the transmission delay circuit provides a time lag between channels with regard to the application timing of the transmission pulse voltage, and focuses the ultrasonic waves generated by a plurality of vibrating elements. It is thus possible to arbitrarily change the focal length by changing the delay time.

The transmission/reception changeover switch performs processing for switching between transmission and reception of ultrasonic waves. The transmission/reception changeover switch protects the reception circuit by preventing pulses with amplitude generated at the time of transmission from being input thereto, and allows signals input at the time of reception to pass to the reception circuit.

The reception delay circuit focuses a receive beam. Since the reflected wave from a given reflector spreads over a spherical surface, the reception delay circuit provides a delay time such that the arrival time at each vibrator is the same, and adds the reflected waves together with consideration for the delay time.

The filter circuit filters reception signals with a bandpass filter and removes noise.

The memory stores reception signals output by the filter circuit, and this function can be realized by an HDD, a memory such as a RAM, or the like.

Here, the ultrasonic probe 200 includes an ultrasonic transducer device.

The ultrasonic transducer device transmits an ultrasonic beam towards an object and receives an ultrasonic echo due to the ultrasonic beam, while scanning the object along a scanning plane. Taking a type of transducer that employs piezoelectric elements as an example, the ultrasonic transducer device has a plurality of ultrasonic transducer elements (ultrasonic element array) and a substrate in which a plurality of openings are arranged in an array. The ultrasonic transducer elements that are used employ a monomorph (unimorph) structure in which a thin piezoelectric element and a metal plate (vibrating film) are stuck together. These ultrasonic transducer elements (vibrating elements) convert electrical vibrations into mechanical vibrations, in which case, warping occurs when the piezoelectric elements expand and contract in-plane, because the size of the metal plate (vibrating film) stuck thereto remains unchanged.

The ultrasonic transducer device may also be configured such that one channel is constituted by a number of ultrasonic transducer elements arranged adjacent to each other, and the ultrasonic beam may be sequentially moved while driving a plurality of channels at one time.

Note that although a type of transducer that uses piezoelectric elements (thin film piezoelectric elements) can be employed as the ultrasonic transducer device, this embodiment is not limited thereto. For example, a type of transducer that uses capacitive elements such as a c-MUT (Capacitive Micromachined Ultrasonic Transducer) or a bulk transducer may be employed. A detailed description of the ultrasonic transducer elements and the ultrasonic transducer device will be given later.

Next, the processing unit 120 performs processing for analyzing the body tissue layers of the subject based on the signals received by the transceiver unit 110.

For example, the processing unit 120 mainly analyzes the amplitude of the received ultrasonic signals, generates data of a B (brightness) mode image (tomographic image) that images the internal structure of the subject, and analyzes the body tissue based on the generated B-mode image data. B-mode image data refers to image data representing the amplitude of an A (Amplitude) mode waveform of the ultrasonic waves as the brightness (luminance) of points. In the case of generating such B-mode image data, the processing unit 120 includes a detection processing unit, a logarithmic transformation processing unit, a gain/dynamic range adjustment unit, a STC (Sensitivity Time Control), and a DSC (Digital Scan Converter), for example.

The detection processing unit performs absolute value (rectification) processing on a reception signal, and thereafter applies a low-frequency pass filter to extract an unmodulated signal.

Next, the logarithmic transformation processing unit performs Log compression on the unmodulated signal, and transforms the representational form so as to facilitate simultaneous checking of the maximum and minimum signal strengths of the reception signal.

The gain/dynamic range adjustment unit then adjusts the signal strength and the area of interest. Specifically, in the gain adjustment processing, a direct current component is added to the signal after Log compression, and in the dynamic range adjustment processing, the signal after Log compression is multiplied by an arbitrary number.

Furthermore, the STC corrects the amplification (brightness) of the signal according to the depth in the body tissue, and acquires an image having uniform brightness across the whole screen.

The DSC then performs scan conversion processing and generates B-mode image data. For example, the DSC converts a line signal into an image signal through interpolation processing such as bilinear interpolation.

Note that the function of the processing unit 120 can be realized by various processors (CPU, etc.), hardware such as ASIC (gate array, etc.), programs, and the like.

The notification information output unit 130 outputs notification information to a notification unit 300, based on a result of the analysis processing. The notification information output unit 130 may be, for example, a communication unit (interface unit) that performs wired or wireless communication with the notification unit 300, an image output unit (image generation unit), or an audio output unit (audio generation unit).

The notification unit (display unit) 300 notifies notification information acquired from the notification information output unit 130. The notification unit 300 may also be a display unit 300, in which case the display unit 300 can be realized by a liquid crystal display, an organic electroluminescence (EL) display, electronic paper or the like, for example.

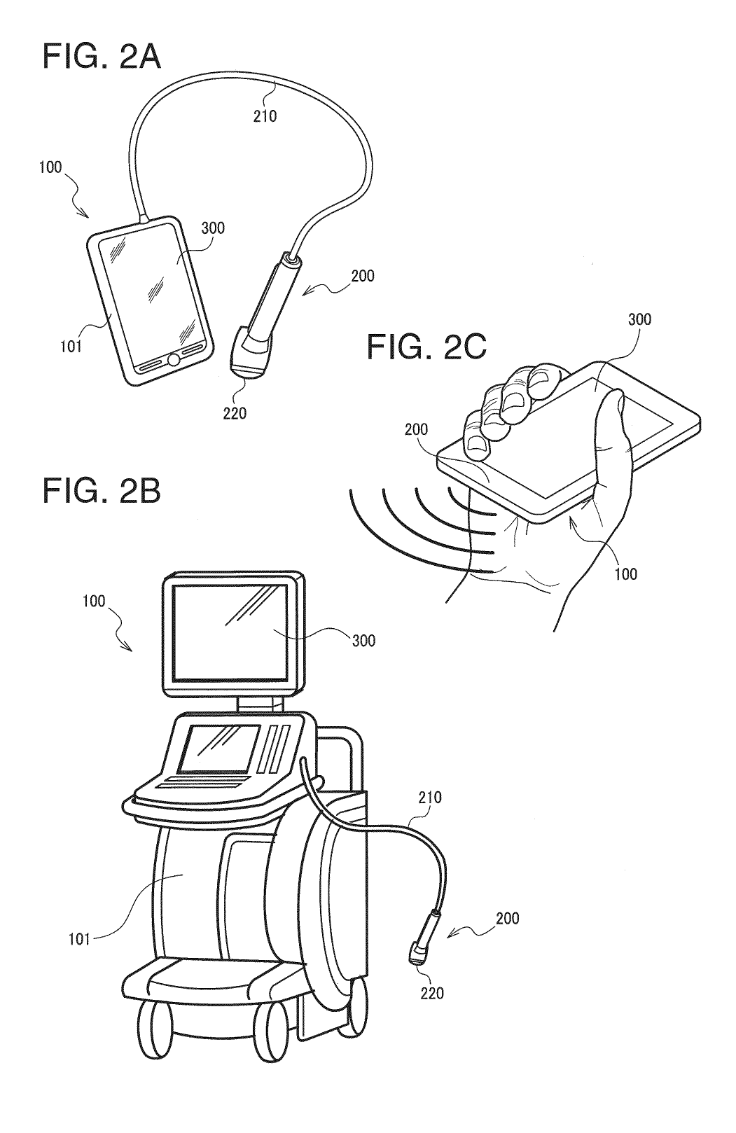

Here, an example of a specific device configuration of the ultrasonic measurement apparatus 100 (broadly, an electronic device) of this embodiment is shown in FIGS. 2A to 2C. FIG. 2A is an example of a handheld ultrasonic measurement apparatus 100, and FIG. 2B is an example of stationary ultrasonic measurement apparatus 100. FIG. 2C is an example of an integrated ultrasonic measurement apparatus 100 that has the ultrasonic probe 200 built into the main body.

The ultrasonic measurement apparatuses 100 of FIGS. 2A and 2B include the ultrasonic probe 200 and a main body 101 of the ultrasonic measurement apparatus (broadly, the main body of an electronic device), and the ultrasonic probe 200 and the main body 101 of the ultrasonic measurement apparatus are connected by a cable 210. Also, a probe head 220 is provided at a tip portion of the ultrasonic probe 200, and the display unit 300 for displaying images is provided in the main body 101 of the ultrasonic measurement apparatus. In FIG. 2C, the ultrasonic probe 200 is built into the ultrasonic measurement apparatus 100 having the display unit 300. In the case of FIG. 2C, the ultrasonic measurement apparatus 100 can be realized by a general-purpose personal digital assistant (PDA) such as a smart phone, for example.

### 3. Details of Processing

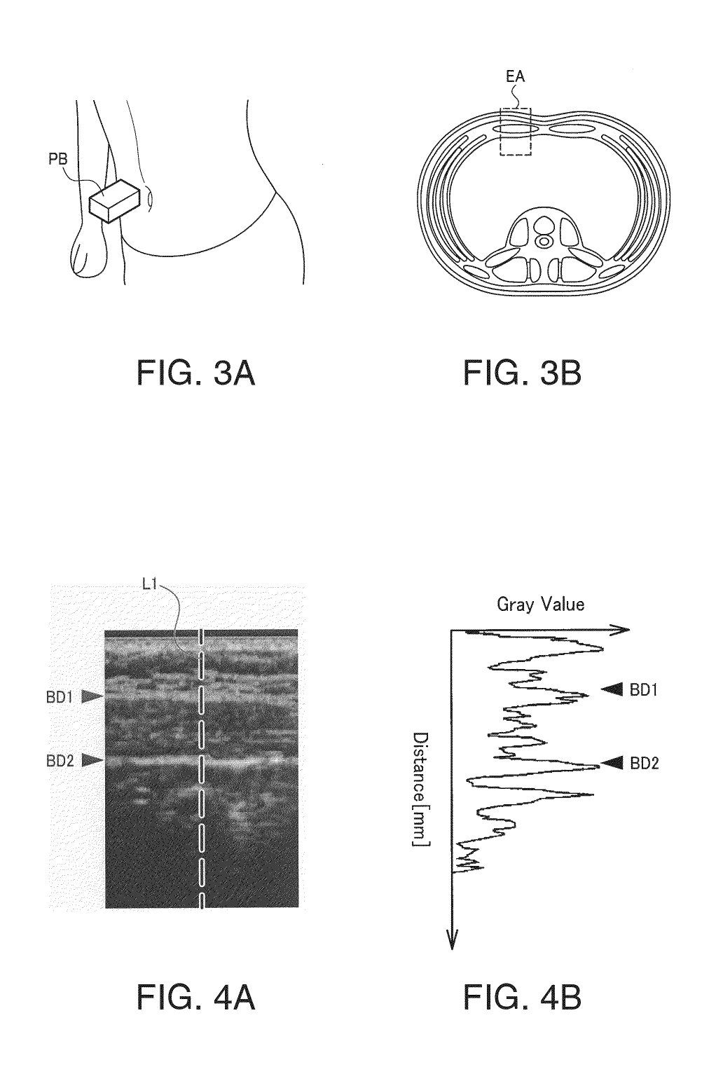

First, the abdomen (rectus abdominis muscle) of a subject (person) is shown in FIG. 3A as an exemplary measurement object of this embodiment, and a cross-sectional view (echo image) of the abdomen of the subject at this time is shown in FIG. 3B. As shown in FIG. 3A, analysis of the body tissue of a measurement area EA shown in FIG. 3B is performed, in the case where an ultrasonic probe PB is pressed against the abdomen.

There are several methods of displaying ultrasonic images, including a method known as B-mode in which the amplitude of ultrasonic waves in a reception signal is converted into luminance values and displayed as a two-dimensional image, and a method known as A-mode in which the amplitude of ultrasonic waves is plotted as a graph. Specifically, an example of a B-mode image is shown in FIG. 4A, and an example of an A-mode waveform is shown in FIG. 4B. In the B-mode image of FIG. 4A, the vertical axis is the depth direction from the upper surface of the subject. On the other hand, the A-mode waveform in FIG. 4B represents the luminance values of a dashed line L1 portion of the B-mode image in FIG. 4A, and the vertical axis represents the distance from the upper surface in the depth direction. Note that although the horizontal axis of a typical A-mode waveform represents the amplitude/strength of reception signals, in the example of FIG. 4B, values obtained by converting the amplitude into luminance values are used, as mentioned above.

A well-informed person will be able to discriminate from the areas that appear white in the B-mode image of FIG. 4A that BD1 is the boundary between the subcutaneous fat layer and the muscle layer and BD2 is the boundary between the muscle layer and internal organs. However, a strong reflection intensity may be obtained from fat accumulation in muscle, diet marks and the like, for example, and portions that appear white other than the boundaries BD1 and BD2 are identifiable, as shown in FIG. 4A, for example. It is thereby difficult to automatically discriminate tissue boundaries by image processing. Since waveform peaks are also formed in portions other than the boundaries BD1 and BD2, a similar problem arises with the A-mode waveform shown in FIG. 4B. In this embodiment, the discrimination accuracy of tissue boundaries is improved in the case where the subject has false boundaries or the like, by performing compositional analysis (measurement of subcutaneous fat layer thickness and muscle thickness of subject) based on B-mode images.

Specifically, ultrasonic waves are emitted onto the subject when a given pressing force is being applied to the subject by the ultrasonic probe and when a pressing force is not being applied, and respective B-mode images are generated. The body tissue is then analyzed based on the B-mode image that is obtained when the given pressing force is being applied and the B-mode image that is obtained when a pressing force is not being applied.

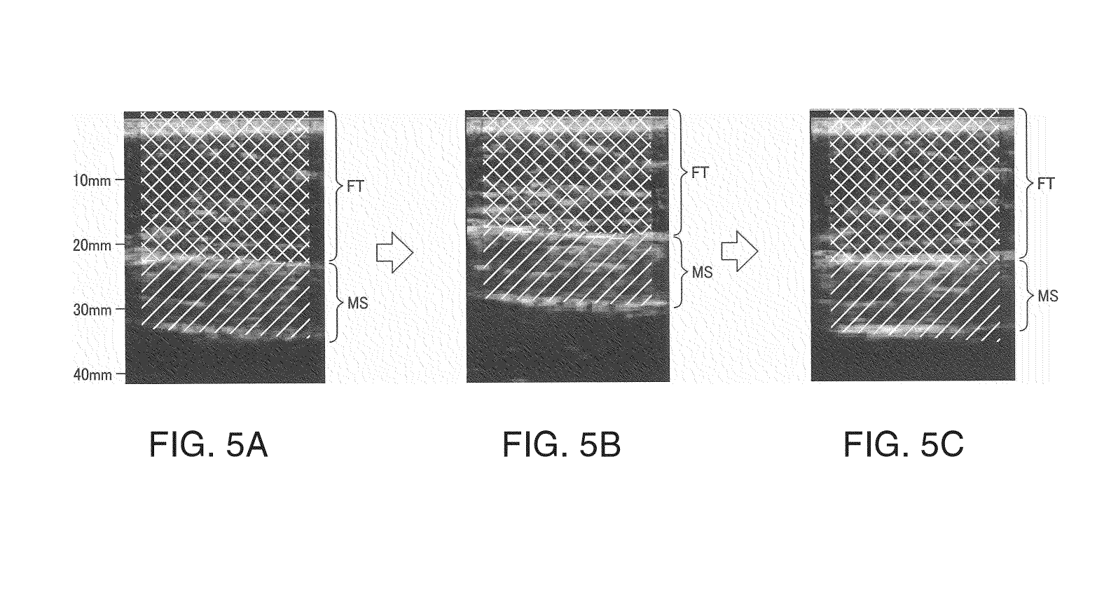

A specific example is shown in FIGS. 5A to 5C. FIGS. 5A to 5C are B-mode images, arranged in time series, that are obtained when transitioning from a state where a pressing force is not applied (FIG. 5A) to a state where a given pressing force is applied to the body tissue measurement site (FIG. 5B) and then back to a state where a pressing force is not applied (FIG. 5C). In FIGS. 5A to 5C, the portion depicted with a net-like pattern is a fat layer FT, and the portion depicted with diagonal lines is a muscle layer MS.

As shown in FIGS. 5A to 5C, it can be confirmed that the thickness of the fat layer FT is reduced under a given pressing force compared with when a pressing force is not applied. On the other hand, the muscle layer MS, while becoming slightly thinner under a given pressing force than when not under a pressing force, does not exhibit as much of a change as the fat layer FT. This is caused by the difference in the properties of the respective body tissues (here, the fat layer and the muscle layer), as discussed above, and it is thus possible to specify the respective body tissues by the difference in the displacement amount by specifying and comparing the displacement amounts when not under a pressing force and when under a pressing force.

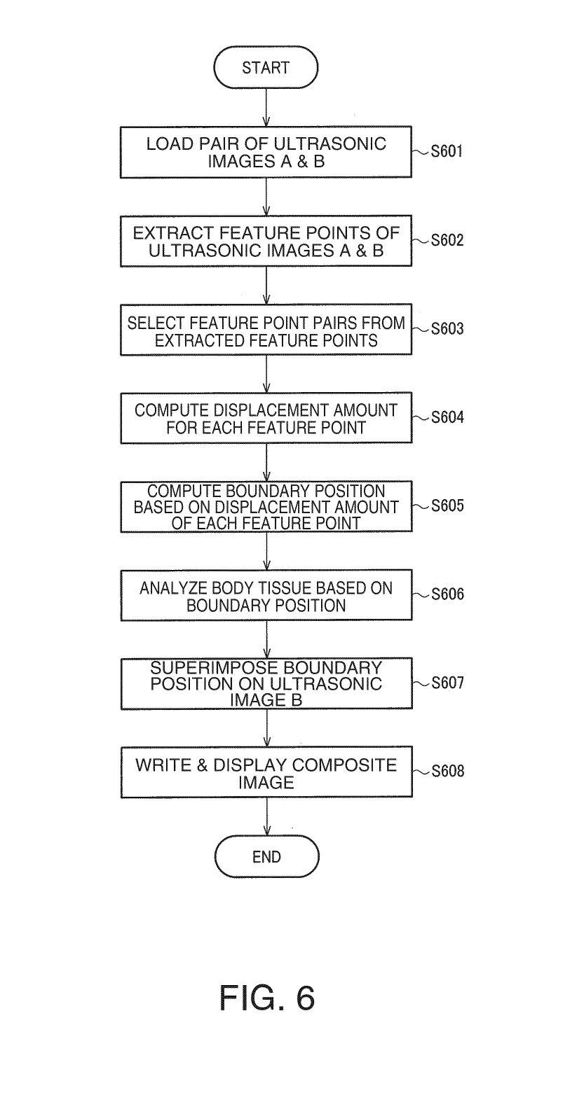

Hereinafter, the processing flow of this embodiment will be described using the flowchart in FIG. 6. Note that, in FIG. 6, description is given from the processing performed after generating the B-mode images. The B-mode images may be generated by an arbitrary method including the aforementioned method. Also, the B-mode images are assumed to be sequentially stored temporarily in the storage unit whenever generated, and images that are required in processing are assumed to be sequentially loaded by the processing unit 120.

First, an ultrasonic image (B-mode image) A and an ultrasonic image B forming a pair are loaded (S601). Here, the ultrasonic image A is a B-mode image obtained when a given pressing force is applied to the subject, and the ultrasonic image B is a B-mode image obtained at a timing before or after the ultrasonic image A when not under a pressing force.

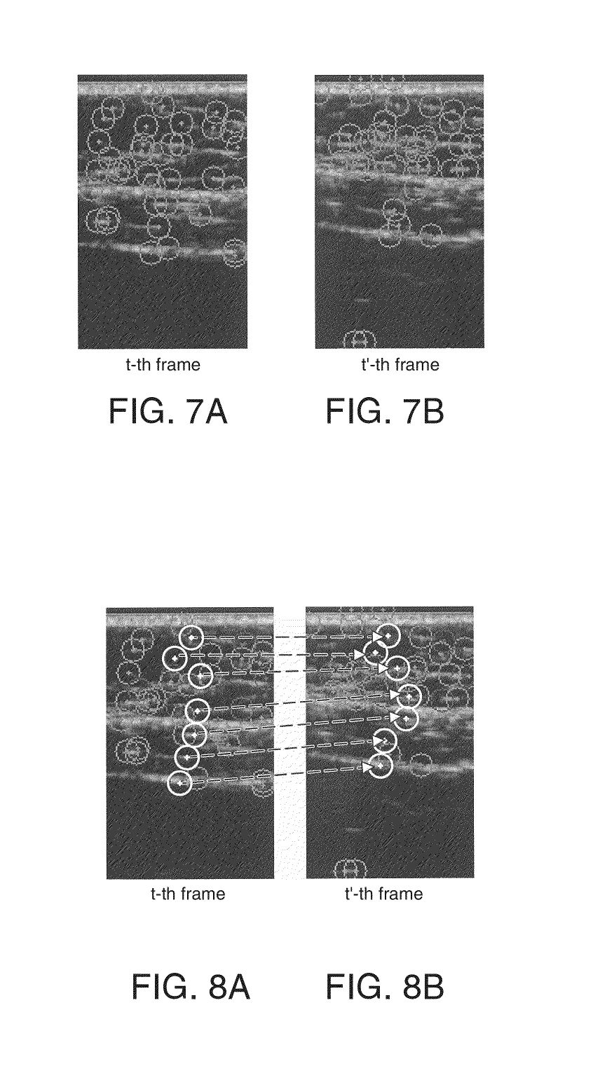

Next, feature points of the ultrasonic image A and the ultrasonic image B are extracted (S602). Feature points are prominent points in the image that are observable. In FIG. 7A the feature points in the B-mode image of the t-th frame when not under a pressing force are marked by circles, and in FIG. 7B the feature points in the B-mode image of the t′-th (=t+Δt) frame when under a given pressing force are marked by circles.

In this example, a corner detection method or the like is used as the method of extracting feature points, but other methods of detecting corner portions (eigenvalues, FAST feature detection) may be used, or local feature descriptors typified by SIFT (Scale-Invariant Feature Transform) as well as SURF (Speeded Up Robust Feature) or the like may be used.

Pairs of mutually corresponding feature points are selected from the group of extracted feature points in the ultrasonic image A and the ultrasonic image B (S603). In other words, a feature point indicating the same site as a feature point in the ultrasonic image A is specified (estimated) from among the group of feature points in the ultrasonic image B, and the two feature points are associated as a pair. In this example, the relationship of corresponding points is specified using RANSAC (RANdom Subject Consensus), but other methods may also be used, such as least-squares, least median of squares or M-estimation.

As a specific example, the result of associating feature points in the ultrasonic image A and the ultrasonic image B is shown in FIGS. 8A and 8B. Here, in the ultrasonic image A shown in FIG. 8A and the ultrasonic image B shown 8B, the associated feature points shown with white circles are connected by arrows. As shown in FIGS. 8A and 8B, not all feature points can necessarily be associated, nor is it necessary to associate all the feature points. The accuracy of subsequent processing for specifying body tissue is, however, improved the greater the number of feature points that can be associated.

Furthermore, the displacement amount is computed for each associated feature point (S604). The displacement amount of a feature point refers to a movement amount representing the extent to which the feature point has moved between adjacent frames. In this example, the average value of a distance change amount Dᵢ of each feature point and other feature points adjacent thereto in the two-dimensional plane of the B-mode image is derived as a displacement amount Δd of the feature point. That is, the displacement amount Δd of a feature point A from the t-th frame to the t′-th (=t+Δt) frame is represented by the following equation (1). Note that n is the number of adjacent feature points and i is a variable between 1 and n inclusive.

$$Δ d = \frac{{\sum }_{i = 1}^{n} {D}_{i}}{n} \qquad ( 1 )$$

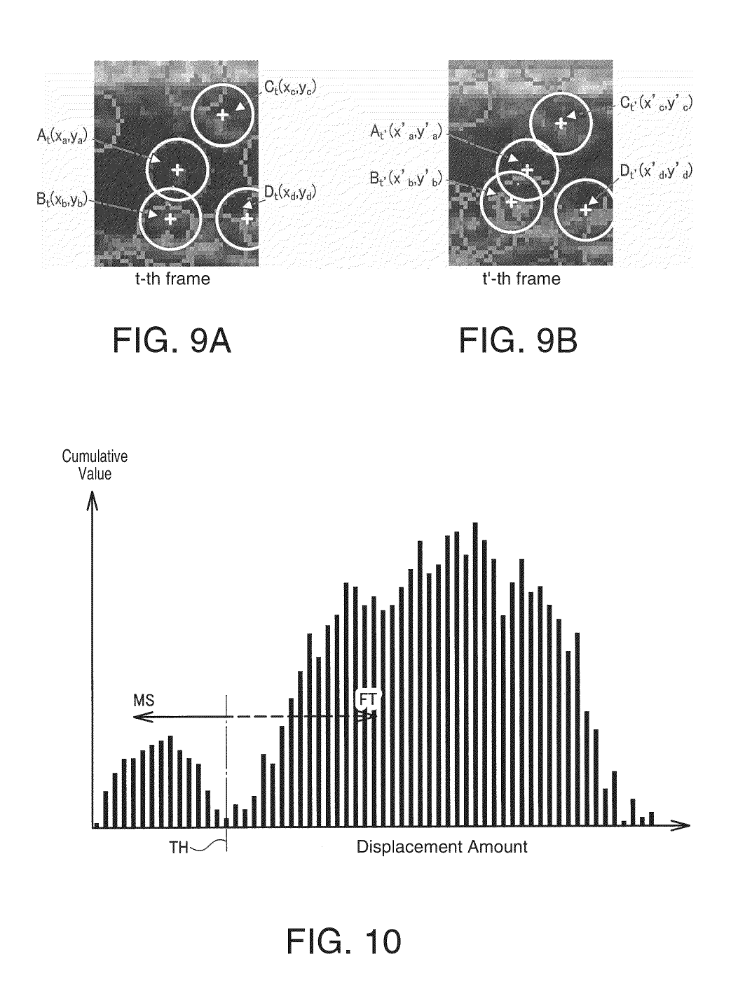

A specific example will be described using FIG. 9A which is an enlarged view of a portion of FIG. 8A, and FIG. 9B which is an enlarged view of a portion of FIG. 8B. FIG. 9A represents feature points (Aₜ, Bₜ, Cₜ, Dₜ) in the fat layer of the t-th frame, and FIG. 9B represents feature points (A_{t′}, B_{t′}, C_{t′}, D_{t′}) in the fat layer of the t′ frame. Also, in FIGS. 9A and 9B, the feature points adjacent to the feature point A are assumed to be the three points B, C and D (n=3). Furthermore, the feature point A_{t′} corresponds to the feature point A_{t′}, and the coordinates of the feature point Aₜ are given as (xₐ, yₐ). A similar relationship applies to the other feature points. At this time, the displacement amount Δd of the feature point A from the t-th frame to the t′-th frame is represented by the following equation (2). Note that D₁, D₂ and D₃ in equation (2) are respectively represented by the following equations (3), (4) and (5).

$$\begin{matrix}Δ d = \frac{{D}_{1} + {D}_{2} + {D}_{3}}{3} & ( 2 ) \\ {D}_{1} = \sqrt{{( {x}_{b} - {x}_{a} )}^{2} + {( {y}_{b} - {y}_{a} )}^{2}} - \sqrt{{( {x}_{b}^{′} - {x}_{a}^{′} )}^{2} + {( {y}_{b}^{′} - {y}_{a}^{′} )}^{2}} & ( 3 ) \\ {D}_{2} = \sqrt{{( {x}_{c} - {x}_{a} )}^{2} + {( {y}_{c} - {y}_{a} )}^{2}} - \sqrt{{( {x}_{c}^{′} - {x}_{a}^{′} )}^{2} + {( {y}_{c}^{′} - {y}_{a}^{′} )}^{2}} & ( 4 ) \\ {D}_{3} = \sqrt{{( {x}_{d} - {x}_{a} )}^{2} + {( {y}_{d} - {y}_{a} )}^{2}} - \sqrt{{( {x}_{d}^{′} - {x}_{a}^{′} )}^{2} + {( {y}_{d}^{′} - {y}_{a}^{′} )}^{2}} & ( 5 )\end{matrix}$$

Note that, here, the displacement amount is calculated using the distance between two points, but the movement amount in only the horizontal direction or the movement amount in only the vertical direction may be calculated and treated as the displacement amount of the feature point.

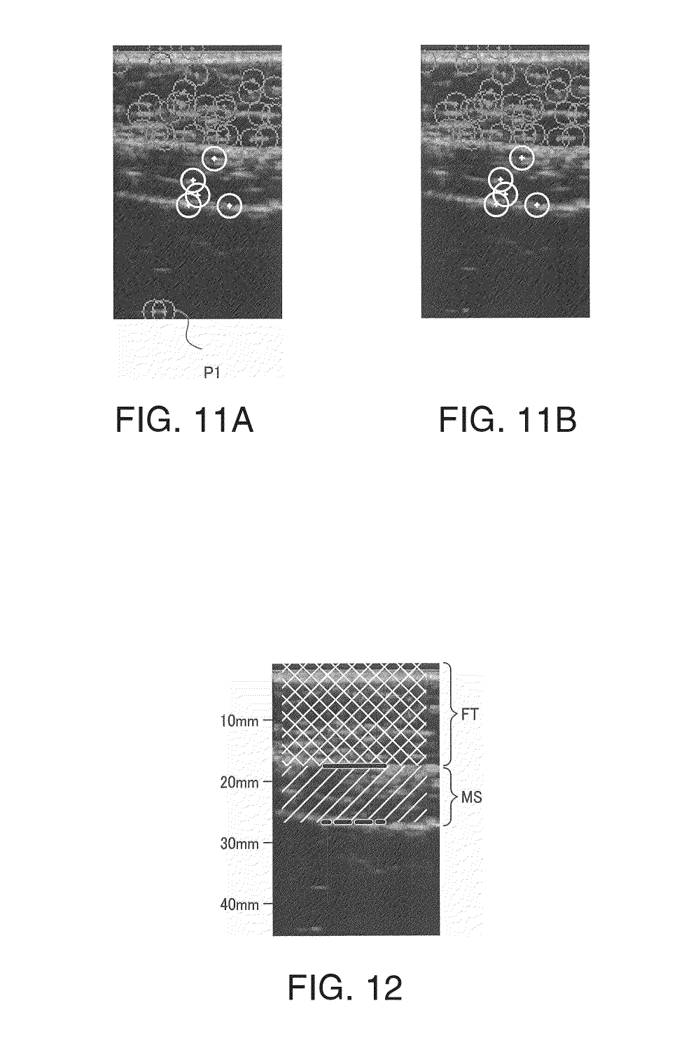

Next, a boundary position is computed based on the displacement amount of each feature point (S605). Specifically, as shown in FIG. 10, a histogram of the displacement amounts for all the feature points is created. It can thus be seen that the histogram has multiple peaks. Here, the subcutaneous fat layer FT and the muscle layer MS are separated based on a given threshold value TH. As discussed above, the displacement amount of the muscle layer MS under a given pressing force is smaller than the displacement amount of the subcutaneous fat layer FT. Thus, in the histogram of FIG. 10, it can be determined that displacement amounts smaller than the threshold value TH are the displacement amounts of a group of feature points of the muscle layer MS which are represented with white circles in FIG. 11A discussed later, and it can be determined that displacement amounts at or above the threshold value TH are the displacement amounts of a group of feature points of the subcutaneous fat layer FT which are represented with gray circles in FIG. 11A discussed later. Note that the threshold value TH is assumed to be computed using methods such as P-tile, mode or discriminant analysis.

Correction processing may be performed by also taking into account positional relationships in a plane and replacing isolated feature points (those surrounded by feature points with different characteristics), rather than using only the displacement amounts of feature points. Specifically, since the feature point detected in a position P1 is located away from other groups of feature points as shown in FIG. 11A, this feature point at the position P1 is determined to be noise and is removed from processing as shown in FIG. 11B.

Next, the body tissue is analyzed based on the boundary position (S606). Here, the distance in the depth (vertical) direction from the top edge of the image to the boundary is measured based on the boundary information obtained as described above. As an example, as shown in FIG. 12, the thickness of the subcutaneous fat layer FT is taken as being the average depth distance in a middle portion (10-mm wide area) of the image from the subcutaneous fat interface to the muscle interface. Also, the thickness of the muscle layer MS is computed using the average depth distance in a middle portion (10-mm wide area) of the image from the muscle interface to an internal organ interface (or bone interface, etc.).

The boundary position information is then superimposed on the ultrasonic image B to generate a composite image (S607), and the composite image is displayed on the display unit (S608).

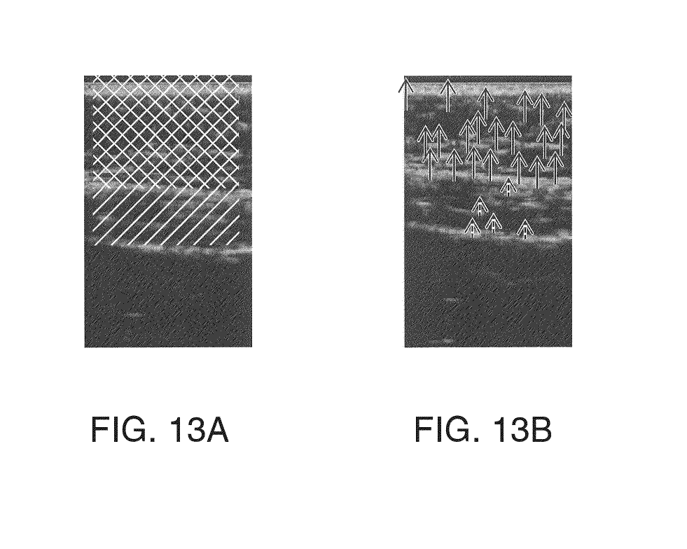

Specifically, FIG. 13A shows an example in which two areas consisting of the subcutaneous fat layer and the muscle layer are displayed with different colors. Note that, in FIG. 13A, for ease of illustration, a net-like pattern is depicted on the area of the fat layer, similarly to FIGS. 5A to 5C, although it is assumed that, in practice, this area is displayed in green. Similarly, although diagonal lines are depicted on the area of the muscle layer, it is assumed that, in practice, this area is displayed in red. This concludes description of the processing flow of the embodiment.

### 4. Methods of the Embodiment

Next, methods employed in this embodiment will be described.

The ultrasonic measurement apparatus 100 of this embodiment includes the transceiver unit 110 for controlling the ultrasonic transducer device with respect to transmission and reception of ultrasonic waves, the processing unit 120 for performing processing for analyzing the body tissue layers of the subject based on signals received by the transceiver unit 110, and the notification information output unit 130 for outputting notification information based on the analysis results. Here, the body tissue layers are deformed within the measurement period. The processing unit 120 then performs analysis processing that involves specifying a plurality of body tissue layers of the subject based on a first reception signal of a first timing within the measurement period and a second reception signal of a second timing within the measurement period, and analyzing the thickness information of each of the specified body tissue layers. Furthermore, the notification information output unit 130 outputs the result of the analysis processing of the thickness information of each body tissue layer as notification information.

In this embodiment, the ultrasonic transducer device is controlled with respect to transmission and reception of ultrasonic waves. The body tissue layer of the subject is deformed within the measurement period, and a plurality of body tissue layers of the subject are specified, based on the first reception signal of the first timing within the measurement period and the second reception signal of the second timing within the measurement period.

Here, body tissue layers refer to the fat layer, the muscle layer, bone, internal organs, blood vessels, and the like, for example.

The first timing refers to an arbitrary timing before the body tissue layers have been deformed, within the measurement period, for example.

Furthermore, the second timing is different from the first timing, and refers to an arbitrary timing after the body tissue layers have been deformed, within the measurement period, for example. The first and second timings are, however, not limited to the aforementioned timings.

Here, the first reception signal refers to a signal obtained when an ultrasonic echo from ultrasonic waves emitted at the first timing is received.

Similarly, the second reception signal refers to a signal obtained when an ultrasonic echo from ultrasonic waves emitted at the second timing is received.

Next, the thickness information of each of the specified body tissue layers is analyzed, and the result of having analyzed the thickness information of each of the specified body tissue layers is output as notification information.

Here, the thickness information may be information representing the thickness, such as the scalar quantity or the vector quantity, for example.

The notification information may be, for example, a numerical value, image data or the like representing the thickness of body tissue or audio data notifying the thickness of body tissue.

The analysis accuracy of the thickness information of body tissue layers can thereby be improved in the case where the subject has false boundaries. The advantage of not having to attach other sensors is also obtained, since processing is performed based only on received ultrasonic signals.

Specifically, the body tissue layers may be deformed by the pressing action of the ultrasonic probe 200.

It is thereby possible, for instance, to detect change when external pressure is applied to body tissue, with a simple operation. As a result, it is possible, for instance, to specify the areas of the different body tissues in the measurement range, based on the characteristics of the respective body tissues under a pressing force.

At the stage at which a signal is received, it is not known what body tissue layers the different portions of the reception signal represent. Thus, it is first necessary to specify the body tissue layers that the different portions of the reception signal represent.

In view of this, the processing unit 120 may derive a group of feature points of the body tissue layers based on the reception signal, and specify a plurality of body tissue layers based on the displacement information of the feature points between the first timing and the second timing.

It is thereby possible, for instance, to derive feature points as points to be observed in the reception signal, and specify what body tissue layers the different portions of the reception signal represent, based on the displacement information of the feature points.

However, the displacement information of feature points cannot be derived if it is not known what feature point an arbitrary feature point at the first timing corresponds to among the group of feature points at the second timing.

In view of this, the processing unit 120 performs processing for associating the feature points in the group of feature points derived based on the first reception signal with the feature points in the group of feature points derived based on the second reception signal, and derives the displacement information of the feature points that are associated in the association processing.

Specifically, the group of feature points derived based on the first reception signal is, for example, the group of feature points shown in the aforementioned ultrasonic image A of FIG. 8A, and the group of feature points derived based on the second reception signal is, for example, the group of feature points shown in the aforementioned ultrasonic image B of FIG. 8B.

It is thereby possible, for instance, to compute the movement amount of each feature point as the displacement information of the feature point between the first timing and the second timing.

Specifically, the processing unit 120 may derive first distance information between a first feature point and a second feature point in the group of feature points derived based on the first reception signal. The processing unit 120 may then derive second distance information between a third feature point associated with the first feature point and a fourth feature point associated with the second feature point in the group of feature points derived based on the second reception signal. Furthermore, the processing unit 120 may derive the displacement information of the first feature point based on difference information between the first distance information and the second distance information.

Described in terms of the aforementioned specific example shown in FIGS. 9A and 9B, for example, the first feature point is the feature point Aₜ, and the second feature point is one of the other feature points located in proximity to the first feature point, such as the feature point Bₜ, for example. In this case, the first distance information is information representing the distance between the feature point Aₜ and the feature point Bₜ.

The third feature point refers to the feature point associated with the first feature point in the aforementioned association processing, among the group of feature points derived based on the second reception signal. The third feature point, although desirably a point representing the exact same site as the first feature point, may be a point representing a different site from the first feature point if the association processing is in error. Specifically, in FIG. 9B, if the first feature point is Aₜ, the third feature point is estimated to be A_{t′}.

Similarly, the fourth feature point refers to the feature point associated with the second feature point in the aforementioned association processing, among the group of feature points derived based on the second reception signal. The fourth feature point may be a point representing a different site from the second feature point if the association processing is in error. Specifically, in FIG. 9B, if the second feature point is Bₜ, the fourth feature point is estimated to be B_{t′}.

In this case, the second distance information is information representing the distance between the feature point A_{t′} and the feature point B_{t′}.

Furthermore, the difference information between the first distance information and the second distance information is derived as shown in the aforementioned equation (3), and the displacement information of the first feature point is derived in accordance with equation (1).

It is thereby possible, for instance, to derive the amount of change in the positional relationship between an arbitrary feature point and another feature point located peripherally to the arbitrary feature point between the first timing and the second timing, as the displacement information of the arbitrary feature point.

Also, as discussed above, the characteristics at the time of deformation are different for each body tissue layer. For example, in the case where a pressing force is applied, the fat layer tends to compress more easily than the muscle layer, whereas bone does not deform under a slight pressing force. Therefore, the displacement information of feature points derived as described above will be similar information for each body tissue layer.

In view of this, the processing unit 120 may perform processing for grouping the displacement information of the feature points, and specify a plurality of body tissue layers based on a result of the grouping.

It is thereby possible, for instance, to specify an area in which a group of feature points sorted into the same group are gathered as the extent of an area occupied by a single body tissue layer.

It is thus possible, for instance, to determine feature points sorted into a different group that are scattered within an area in which a group of feature points sorted into the same group are gathered, as shown in FIG. 11A, as being intramuscular fat or diet marks, and to treat such an area as the fat layer or the muscle layer (remove such feature points from processing).

The processing unit 120 may also create a histogram of the displacement information of the feature points, and perform processing for grouping the displacement information based on the created histogram.

Specifically, the processing unit 120 may create a histogram such as shown in FIG. 10.

It is thereby possible, for instance, to accurately specify each body tissue layer, even in the case where a luminance peak does not occur at the boundary between body tissue layers.

To summarize the processing flow up until this point, the processing unit 120 may derive a first group of feature points of a body tissue layer based on the first reception signal and a second group of feature points of a body tissue layer based on the second reception signal. The processing unit 120 may then perform processing for associating the feature points in the first group of feature points with the feature points in the second group of feature points, and derive the displacement information of the feature points that are associated in the association processing. Furthermore, the processing unit 120 may perform processing for grouping the derived displacement information, specify a plurality of body tissue layers based on the grouping result, and analyze the thickness information of each of the specified body tissue layers.

It is thereby possible, for instance, to improve the analysis accuracy of the thickness information of body tissue layers, in the case where the subject has false boundaries.

Next, a method of notifying notification information will be described.

First, the notification information output unit 130 may generate, as notification information, an image representing a first body tissue layer and a second body tissue layer among the plurality of body tissue layers specified by the processing unit 120 with different pixel values.

Here, the first body tissue layer is assumed to be the fat layer, for example, and the second body tissue layer is assumed to be the muscle layer, for example.

It is thereby possible, for instance, for the display unit 300 to display an image representing the first body tissue layer and the second body tissue layer with different pixel values.

Furthermore, the notification information output unit 130 may generate, as notification information, an image representing the first body tissue layer and the second body tissue layer among the plurality of body tissue layers specified by the processing unit 120 with different colors or brightnesses.

It is thereby possible, for instance, for the display unit 300 to display an image representing the first body tissue layer and the second body tissue layer with different colors or brightnesses.

The display mode of areas representing the respective body tissues may be changed according to the displacement amount. For example, luminance values (or combinations of hue, saturation, brightness, etc.) may be increased or decreased according to the size of the displacement amount.

The notification information output unit 130 may also generate, as notification information, an image representing the amount of change in the thickness of each body tissue layer between the first timing and the second timing.

Here, an image representing the amount of change in the thickness of each body tissue layer between the first timing and the second timing refers to, for example, vector information such as indicated with arrows in FIG. 13B. The vectors in FIG. 13B are vectors from the position of the feature points before deformation to the position of the feature points after deformation. Also, the image representing the amount of change in the thickness of each body tissue layer between the first timing and the second timing does not necessarily have to be vectors, and arrows that differ according to the displacement amount, or the like, may be displayed.

It is thereby possible, for instance, for the display unit to display an image representing the amount of change in the thickness of each body tissue layer between the first timing and the second timing. Therefore, it is possible, for instance, to represent the displacement amount of feature points by qualities such as the color, thickness and length of vectors.

The subject may move his or her body tissue with the ultrasonic probe 200 lightly pressed against the body tissue, and the aforementioned measurement processing may be performed before and after this action. For example, the action of bending and extending an arm may be performed with the ultrasonic probe 200 pressed against a front portion of the upper arm, and the amount of displacement in the expansion and contraction of the body tissue with the arm in the extended state and the bent state may be calculated. Here, only the amount of horizontal movement is taken into consideration. Alternatively, in the case where the vertical component is also used in addition to the horizontal component, the displacement amount may be calculated after increasing the weight of the horizontal component.

The ultrasonic measurement apparatus of this embodiment discriminates the body tissue of a subject based on qualities such as hardness, softness and the like representing tissue characteristics. Given this, application in fields where noninvasive measurement and analysis is required, such as healthcare, farming (evaluation of meat quality/quantity), and fisheries (evaluation of fish quality/quantity), for example, can also be anticipated.

Note that the processing performed by the ultrasonic measurement apparatus 100, the ultrasonic imaging apparatus 400 and the like of this embodiment may also be partly or mostly realized by a program. In this case, the ultrasonic measurement apparatus 100, the ultrasonic imaging apparatus 400 and the like of this embodiment are realized by a processor such as a CPU executing the program. Specifically, a program stored in an information storage medium is read out, and a processor such as a CPU executes the read program. Here, the information storage medium (computer-readable medium) is a medium for storing programs, data and the like, and this function can be realized by an optical disk (DVD, CD, etc.), an HDD (hard disk drive), or a memory (card memory, ROM, etc.). A processor such as a CPU then performs the various processing of this embodiment based on a program (data) stored in the information storage medium. That is, a program for causing a computer (an apparatus provided with an operation unit, a processing unit, a storage unit, and an output unit) to function as the constituent elements of this embodiment (a program for causing a computer to execute the processing of the constituent elements of this embodiment) is stored in the information storage medium.

### 5. Ultrasonic Transducer Elements

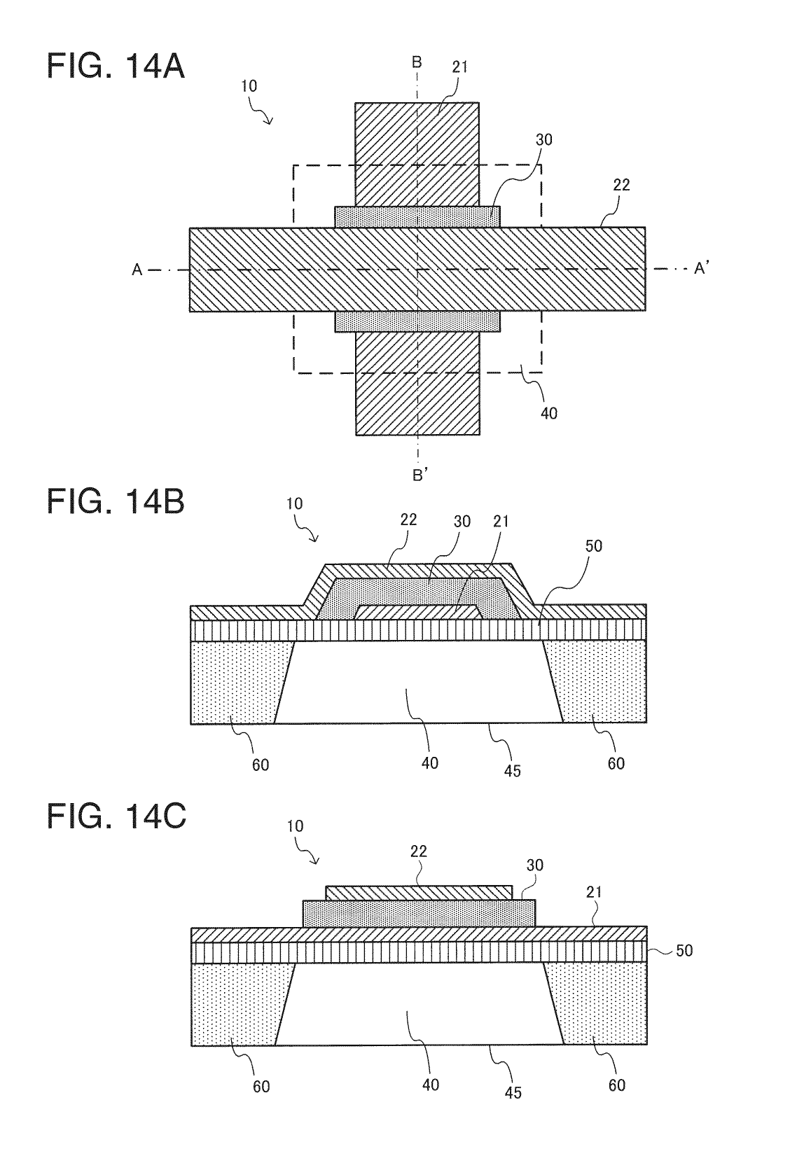

An exemplary configuration of an ultrasonic transducer element 10 of the ultrasonic transducer device is shown in FIGS. 14A to 14C. This ultrasonic transducer element 10 has a vibrating film (membrane, supporting member) 50 and a piezoelectric element portion. The piezoelectric element portion has a first electrode layer (lower electrode) 21, a piezoelectric layer (piezoelectric film) 30, and a second electrode layer (upper electrode) 22.

FIG. 14A is a plan view of the ultrasonic transducer element 10 formed on a substrate (silicon substrate) 60 seen from a direction perpendicular to an element forming surface side of the substrate 60. FIG. 14B is a cross-sectional view showing a cross-section along A-A′in FIG. 14A. FIG. 14C is a cross-sectional view showing a cross-section along B-B′ in FIG. 14A.

The first electrode layer 21 is formed with a metal thin film, for example, on top of the vibrating film 50. This first electrode layer 21 may be an interconnect that extends to outside the element forming area as shown in FIG. 14A and is connected to adjacent ultrasonic transducer elements 10.

The piezoelectric layer 30 is formed of a PZT (lead zirconate titanate) thin film, for example, and is provided so as to cover at least part of the first electrode layer 21. Note that the material of the piezoelectric layer 30 is not limited to PZT, and lead titanate (PbTiO₃), lead zirconate (PbZrO₃), lead lanthanum titanate ((Pb, La)TiO₃) or the like may be used, for example.

The second electrode layer 22 is formed with a metal thin film, for example, and is provided so as to cover at least part of the piezoelectric layer 30. This second electrode layer 22 may be an interconnect that extends outside the element forming area as shown in FIG. 14A and is connected to adjacent ultrasonic transducer elements 10.

The vibrating film (membrane) 50 is provided so as to close off an opening 40 with a two-layer structure consisting of an SiO₂ thin film and a ZrO₂ thin film, for example. This vibrating film 50 is able to support the piezoelectric layer 30 and the first and second electrode layers 21 and 22, and to generate ultrasonic waves through vibration in accordance with the expansion and contraction of the piezoelectric layer 30.

The opening 40 is formed by etching from the back surface (surface on which elements are not formed) side of the substrate 60 (silicon substrate) using reactive ion etching (RIE) or the like. The resonance frequency of the ultrasonic waves is determined by the size of an open portion 45 of this opening 40, and ultrasonic waves are irradiated from the piezoelectric layer 30 side (from the far side to the near side in FIG. 14A).

The lower electrode (first electrode) of the ultrasonic transducer element 10 is formed by the first electrode layer 21, and the upper electrode (second electrode) is formed by the second electrode layer 22. Specifically, the portion of the first electrode layer 21 that is covered by the piezoelectric layer 30 forms the lower electrode, and the portion of the second electrode layer 22 that covers the piezoelectric layer 30 forms the upper electrode. That is, the piezoelectric layer 30 is sandwiched between the lower electrode and the upper electrode.

### 6. Ultrasonic Transducer Device

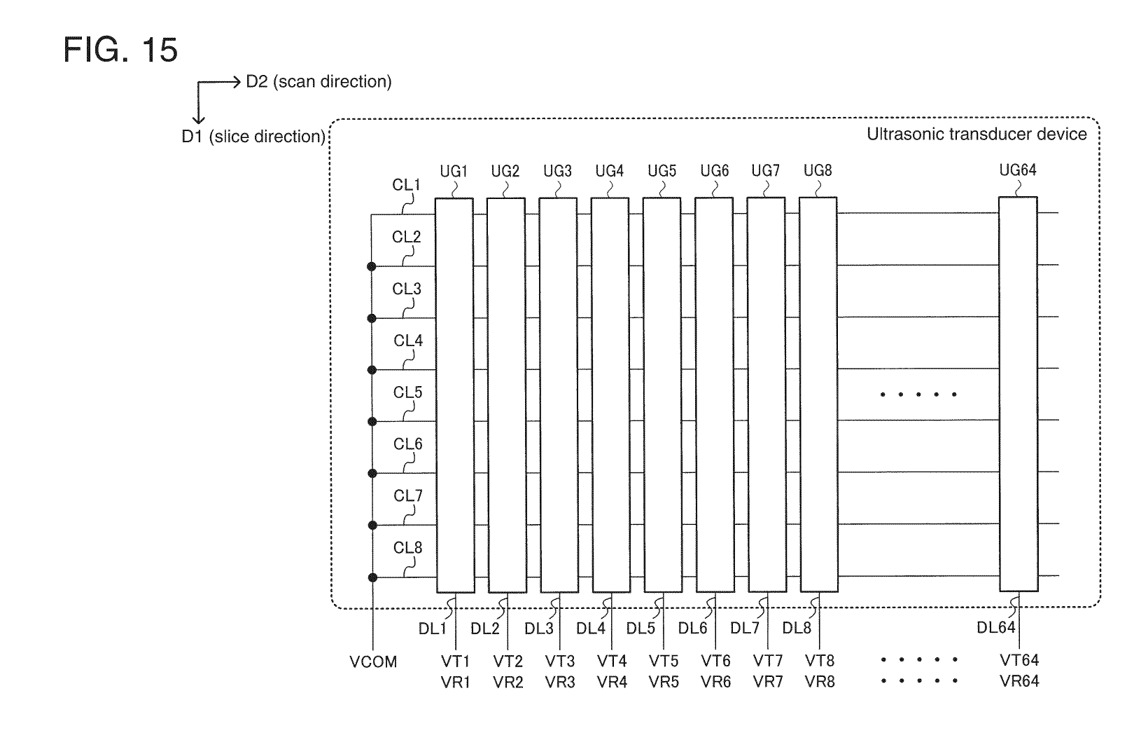

FIG. 15 shows an exemplary configuration of an ultrasonic transducer device (element chip). The ultrasonic transducer device having this exemplary configuration includes a plurality of ultrasonic transducer element groups UG1 to UG64, drive electrode lines DL1 to DL64 (broadly, 1st to n-th drive electrode lines, where n is an integer of 2 or more), and common electrode lines CL1 to CL8 (broadly, 1st to m-th common electrode lines, where m is an integer of 2 or more). Note that the number (n) of drive electrode lines and the number (m) of common electrode lines are not limited to the number shown in FIG. 15.

The plurality of ultrasonic transducer element groups UG1 to UG64 are arranged in 64 columns in a second direction D2 (scanning direction). Each of the ultrasonic transducer element groups UG1 to UG64 has a plurality of ultrasonic transducer elements arranged in a first direction D1 (slice direction).

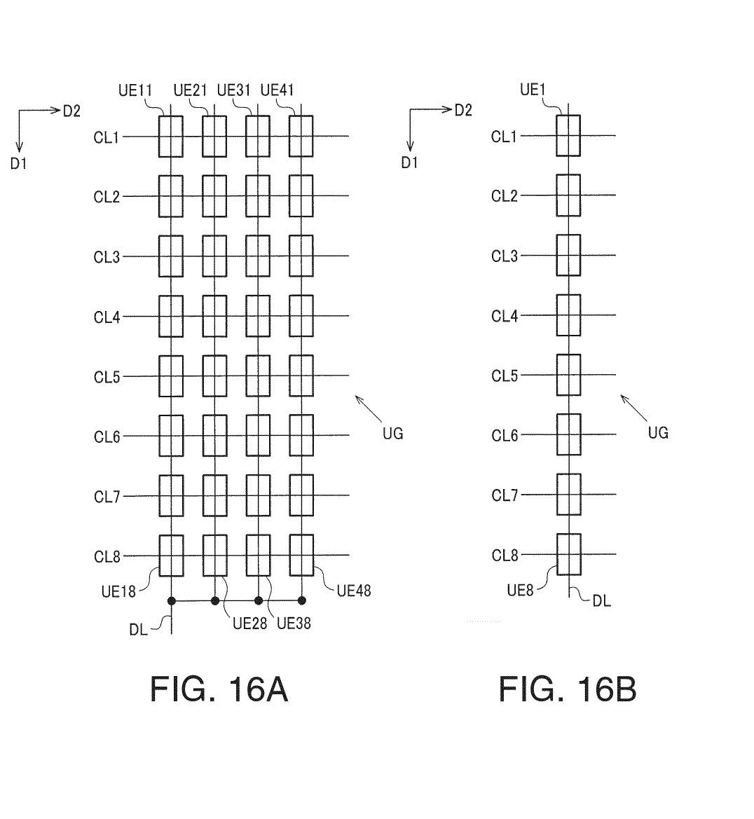

FIG. 16A shows an exemplary ultrasonic transducer element group UG (UG1 to UG64). In FIG. 16A, the ultrasonic transducer element group UG is constituted by first to fourth element columns. The first element column is constituted by ultrasonic transducer elements UE11 to UE18 arranged in the first direction D1, and the second element column is constituted by ultrasonic transducer elements UE21 to UE28 arranged in the first direction D1. The third element column (UE31 to UE38) and the fourth element column (UE41 to UE48) are also similarly constituted. A drive electrode line DL (DL1 to DL64) is connected in common to these first to fourth element columns. Also, the common electrode lines CL1 to CL8 are connected to the ultrasonic transducer elements of the first to fourth element columns.

The ultrasonic transducer element group UG in FIG. 16A constitutes one channel of the ultrasonic transducer device. That is, the drive electrode line DL is equivalent to the drive electrode line of one channel, and the transmission signal for one channel from the transmission circuit is input to the drive electrode line DL. Also, the reception signal of one channel from the ultrasonic transducer element group UG is output from the drive electrode line DL. Note that the number of element columns constituting one channel is not limited to four columns as shown in FIG. 16A, and may be fewer than four columns or more than four columns. For example, there may be one element column, as shown in FIG. 16B.

As shown in FIG. 15, the drive electrode lines DL1 to DL64 (1st to n-th drive electrode lines) are laid out in the first direction D1. The j-th (j being an integer where 1≦j≦n) drive electrode line DLj (j-th channel) of the drive electrode lines DL1 to DL64 is connected to the first electrode (e.g., lower electrode) of the ultrasonic transducer elements of the j-th ultrasonic transducer element group UGj.

Transmission signals VT1 to VT64 are supplied to the ultrasonic transducer elements via the drive electrode lines DL1 to DL64 in a transmission period during which ultrasonic waves are emitted. Also, reception signals VR1 to VR64 from the ultrasonic transducer elements are output via the drive electrode lines DL1 to DL64 in a reception period during which ultrasonic echo signals are received.

The common electrode lines CL1 to CL8 (1st to m-th common electrode lines) are laid out in the second direction D2. The second electrode of the ultrasonic transducer elements is connected to one of the common electrode lines CL1 to CL8. Specifically, as shown in FIG. 15, for example, the i-th (i being an integer where common electrode line CLi of the common electrode lines CL1 to CL8 is connected to the second electrode (e.g., upper electrode) of the ultrasonic transducer elements arranged in the i-th row.

A common voltage VCOM is supplied to the common electrode lines CL1 to CL8. This common voltage VCOM need only be a constant direct current voltage, and may be other than 0V, that is, other than ground potential.

In the transmission period, a difference voltage between the transmission signal voltage and the common voltage is applied to the ultrasonic transducer elements, and ultrasonic waves having a predetermined frequency are irradiated.

Note that the arrangement of the ultrasonic transducer elements is not limited to the matrix arrangement shown in FIG. 15, and a staggered arrangement or the like can be applied.

Also, in FIGS. 16A and 16B, the case where a single ultrasonic transducer element is used as both a transmitting element and a receiving element is shown, although this embodiment is not limited thereto. For example, ultrasonic transducer elements for use as transmitting elements and ultrasonic transducer elements for use as receiving elements may be separately provided and arranged in an array.

Although this embodiment has been described in detail above, the person skilled in the art will appreciate that numerous modifications that do not deviate substantially from the novel aspects and effects of the invention are possible. Accordingly, such modifications are also intended to be included within the scope of the invention. For example, terms that appear in the description or drawings at least once together with other broader or synonymous terms can be replaced by those other terms at any location within the description or drawings. Also the configurations and operations of the ultrasonic measurement apparatus and the ultrasonic imaging apparatus are not limited to those described in this embodiment, and various modifications can be implemented.

The entire disclosure of Japanese Patent Application No. 2013-065760, filed Mar. 27, 2013 is expressly incorporated by reference herein.

What is claimed is:

1. An ultrasonic measurement apparatus comprising:
a transceiver unit that controls an ultrasonic transducer device with respect to transmission and reception of ultrasonic waves;
a processing unit that performs processing for analyzing a body tissue layer of a subject based on a signal received by the transceiver unit; and
a notification information output unit that outputs notification information based on a result of the analysis processing,
wherein the processing unit performs the analysis processing by specifying a plurality of body tissue layers of the subject, based on a first reception signal of a first timing within a measurement period and a second reception signal of a second timing within the measurement period at which a deformation state of the body tissue layers differs from the deformation state at the first timing, and analyzing thickness information of each of the specified body tissue layers, and
the notification information output unit outputs the result of the analysis processing of the thickness information of each body tissue layer as the notification information.

2. The ultrasonic measurement apparatus according to claim 1, wherein the processing unit derives a group of feature points of the body tissue layers based on the reception signals, and specifies the plurality of body tissue layers, based on the displacement information of the feature points between the first timing and the second timing.

3. The ultrasonic measurement apparatus according to claim 2, wherein the processing unit derives first distance information between a first feature point and a second feature point in the group of feature points derived based on the first reception signal, derives second distance information between a third feature point associated with the first feature point and a fourth feature point associated with the second feature point in the group of feature points derived based on the second reception signal, and derives the displacement information of the first feature point, based on difference information between the first distance information and the second distance information.

4. The ultrasonic measurement apparatus according to claim 2, wherein the processing unit performs processing for grouping the displacement information of the feature points, and specifies the plurality of body tissue layers based on a result of the grouping.

5. The ultrasonic measurement apparatus according to claim 4, wherein the processing unit creates a histogram of the displacement information of the feature points, and performs the processing for grouping the displacement information based on the created histogram.

6. The ultrasonic measurement apparatus according to claim 2, wherein the processing unit performs processing for associating the feature points in the group of feature points derived based on the first reception signal with the feature points in the group of feature points derived based on the second reception signal, and derives the displacement information of the feature points that are associated in the association processing.

7. The ultrasonic measurement apparatus according to claim 1, wherein the processing unit derives a first group of feature points of the body tissue layers based on the first reception signal and a second group of feature points of the body tissue layers based on the second reception signal, performs processing for associating the feature points in the first group of feature points with the feature points in the second group of feature points, derives the displacement information of the feature points that are associated in the association processing, performs processing for grouping the derived displacement information and specifies the plurality of body tissue layers based on a result of the grouping, and analyzes the thickness information of each of the specified body tissue layers.

8. The ultrasonic measurement apparatus according to claim 1, wherein the notification information output unit generates, as the notification information, an image representing a first body tissue layer and a second body tissue layer among the plurality of body tissue layers specified by the processing unit with different pixel values.

9. The ultrasonic measurement apparatus according to claim 1, wherein the notification information output unit generates, as the notification information, an image representing a first body tissue layer and a second body tissue layer among the plurality of body tissue layers specified by the processing unit with different colors or brightnesses.

10. The ultrasonic measurement apparatus according to claim 1, wherein the notification information output unit generates, as the notification information, an image representing an amount of change in a thickness of each body tissue layer between the first timing and the second timing.

11. The ultrasonic measurement apparatus according to claim 1, wherein the deformation states of the body tissue layers at the first timing and the second timing within the measurement period differ due to a pressing action of an ultrasonic probe.

12. An ultrasonic imaging apparatus comprising:
the ultrasonic measurement apparatus according to claim 1; and
a display unit that displays the notification information.

13. An ultrasonic measurement method comprising:
controlling an ultrasonic transducer device with respect to transmission and reception of ultrasonic waves;
specifying a plurality of body tissue layers of a subject, based on a first reception signal of a first timing within a measurement period and a second reception signal of a second timing within the measurement period at which a deformation state of the body tissue layers differs from the deformation state at the first timing;
analyzing thickness information of each of the specified body tissue layers; and
outputting a result of having analyzed the thickness information of each body tissue layer as notification information.
